(19) United States
(12) Patent Application Publication
(10) Pub. No.: US 2004/0176705 A1
(43) Pub. Date: Sep. 9, 2004

(54) Cartridge having an integrated collection element for point of care system

(76) Inventors: Timothy A. Stevens, Warwick, NJ (US); C. Mark Newby, Tuxedo, NY (US); Bradley M. Wilkinson, North Haledon, NJ (US); Robert G. Ellis, Wykoff, NJ (US); Charles G. Hwang, Ridgewood, NJ (US); Frank Francavilla, Newton, NJ (US); Jamieson W. Crawford, New York, NY (US)

Correspondence Address: Scott J. Rittman, Esq., Becton, Dickinson and Company, 1 Becton Drive, Franklin Lakes, NJ, 07417-1880, US

(21) Appl. No.: 10/377,621

(22) Filed: Mar. 4, 2003

(51) Int. Cl. A61B 5/00

(52) U.S. Cl. 600/584

(57) ABSTRACT

A system is provided which may be used for collecting and testing fluid samples at a patient point of care location. A disposable collection and containment device is provided for collecting a fluid sample directly into a containment chamber containing testing mechanisms using an integrated collection mechanism for immediate evaluation of the collected sample. The integrated collection cartridge contains an array of electrical contacts, electrochemical sensors and circuitry configured to electrically couple with an analytical device, such as a personal digital assistant (PDA) or a stand-alone computer workstation, which controls the testing of the fluid sample within the cartridge and provides a rapid indication of test results at the point of care.

### CROSS-REFERENCE TO RELATED APPLICATIONS

Related subject matter is disclosed in a U.S. patent application Ser. No. ______ of Stevens et al. entitled “Collection Device Adapted To Accept Cartridge For Point Of Care System”, Attorney Docket No. 43698, filed concurrently, the entire contents of which being incorporated herein by reference.

### FIELD OF THE INVENTION

The present invention relates to a system for capturing and testing fluid samples at a patient point of care location. In particular, the present invention relates to a system for collecting a fluid sample such as blood, directly into a sealed cartridge which includes an integrated collection mechanism for collecting the sample, and a testing mechanism for evaluation of the collected sample immediately upon collection. The cartridge is further configured to safely shield the collection mechanism after collection if required. The cartridge contains an array of electrical contacts, electrochemical sensors (i.e., biosensor chips) and circuitry configured to electrically couple with a remote analytical device, such as a hand-held personal digital assistant (PDA), or a stand-alone computer workstation, which controls testing of the fluid sample within the cartridge and provides a rapid indication of test results at the point of care.

### BACKGROUND OF THE INVENTION

In a typical healthcare environment, clinical laboratories often perform numerous tests for doctors and healthcare professionals. Such laboratories perform these tests on various fluid samples, such as human blood, urine, plasma, serum or other fluids in order to measure chemical or physical properties of the samples. The results of these tests are used by doctors and healthcare professionals to make clinical decisions related to patient care and treatment. Because such results are used to make decisions for patient care, dependable test results are of the utmost importance. However, in addition to dependability considerations, many situations may require immediate determination of test results for effective care and treatment. In such cases, remote laboratory facilities are often unable to provide test results in a useful manner. As pointed out in U.S. Pat. No. 5,096,669 issued to Lauks et al., the entire content of which is incorporated herein by reference, many situations require test results immediately, such as in the physician's office, hospital emergency room or at the patient's bedside.

In any sample testing scenario, the first consideration typically concerns sample collection and thereafter, sample transfer to a testing facility or apparatus. As discussed in U.S. Pat. No. 6,074,383 issued to Grippi et al., the entire content of which is incorporated herein by reference, the taking of samples such as blood, is considered a necessary part of the process of diagnosing and controlling many forms of disease. As described in the Grippi patent, blood samples are obtained by puncturing the skin of a patient's finger with a sharp object such as a syringe or pointed blade which are typically disposable, such that once used, each may be discarded. Details of syringe construction and use in sample collection are described in U.S. Pat. No. 6,196,998 issued to Jansen et al., the entire content of which is incorporated herein by reference. Such conventional syringes described in the Jansen Patent include a barrel having an open distal end, typically engaged to a needle assembly with a needle cannula, and an opposed proximal end with a cylindrical wall extending between ends and defining a substance retaining chamber. As may be appreciated by those skilled in the art, collection of a sample within the retaining chamber of the syringe merely requires needle insertion at the distal end, and a sliding movement of a plunger within the chamber from the proximal end.

One alternative to the syringe as a blood sample collection device is discussed in the Grippi patent referenced above. Lasers, commonly known as laser lancets, may be used as a substitute for a needle or pointed blade for obtaining blood samples from patients. A laser lancet, as with a mechanical lancet, can be used to puncture the surface of the skin for exposing blood samples where the blood may then be collected for analysis.

As pointed out in U.S. Pat. No. 6,221,307 issued to Norman J. Hutton, the entire content of which is incorporated herein by reference, the collected blood samples may be taken and analyzed in hospital or clinical situations for various medical purposes. Sample analysis may include detection of pH, pCO₂, pO₂, Na⁺, Ca⁺⁺, K⁺, hematocrit and glucose levels in the sample, in addition to sample temperature measurements through the use of real time sensors such as those described in U.S. Pat. No. 5,212,050 issued to Mier et al., and in U.S. Pat. No. 5,200,051 issued to Cozzette et al., the entire content of each being incorporated herein by reference.

Collection, handling and testing of these samples typically requires the use of various medical testing instruments and, as pointed out in the Hutton patent referenced above, collection ideally occurs using standard sized collection devices. The use of standard sized collection devices allows the design and use of testing instruments which are configured to process samples without removal from the collection device. One such form of testing instrument currently available is a hand held analyzer, which may be configured to accept samples contained within a standard collection device. Hand-held analyzers for sample testing are extensively discussed in U.S. Pat. No. 6,066,243 issued to Anderson et al., and in U.S. Patent Application Publication No. U.S. 2002/0002326 issued to Causey et al., the entire content of each being incorporated herein by reference. Many such analyzers are configured to accept samples for testing via access ports adapted to receive small containment cartridges containing the sample for evaluation. Analyzers such as PDA-based devices are very cost effective, easily upgraded and allow on the spot analysis. Additional details regarding such configurations are discussed in U.S. Pat. No. 5,096,669, referenced above, in an article by Jason Thibeault entitled “Move Toward PDA-Based Devices Gets Boost from FDA”, Medical Device & Diagnostic Industry, August 2002, in an article by Ian Austin entitled “Palmtops In The Operating Room”, New York Times, Aug. 22, 2002, and in an article by Stephanie De Ritis entitled “Expanding Exceeding POCT Boundaries”, Advance/Laboratory, August 2002, the entire content of each being incorporated herein by reference.

The containment cartridge method of sample collection and testing has proved successful in many applications. Containment cartridges include a small containment chamber into which a fluid sample is placed for testing, typically via a capillary tube placed into contact with an exposed fluid sample source. The chamber includes an extensive sensor array, such that numerous tests and evaluations may be performed on the contained sample. The cartridge is built as a standardized package which is configured to fit within an access port on a testing device that electrically couples to the sensor array of the cartridge, and directly collects information from the sensors regarding the contained sample. The move to standardize devices for interfacing between workstations and clinical systems is discussed further in the article entitled “Expanding Exceeding POCT Boundaries”, referenced above. One such containment cartridge compatible with a hand-held analyzer is discussed in U.S. Pat. No. 5,638,828 issued to Lauks et al., the entire content of which is incorporated herein by reference, and in U.S. Pat. No. 5,096,669 also issued to Lauks et al. and referenced above. The Lauks patent discloses a collection device for collecting a volume of blood or other fluids in a capillary tube housed within a sealed cartridge for diagnostic testing using a hand-held analytical device. As discussed in the Lauks patent, a fluid sample is introduced into a disposable cartridge through an orifice at one end of the cartridge. The sample enters the cartridge by putting the orifice in contact with an exposed source and a sample is drawn by capillary action into a conduit within the cartridge.

However, the cartridge disclosed in the Lauks patent requires that a medical professional first prick the patient's finger with a finger stick to draw a small amount of blood through a dermal puncture. The medical professional is then required to place the orifice of the cartridge in contact with the blood sample formed on the dermal puncture in the patient's finger to draw the blood into the conduit of the collection cartridge. This method requires an exposed fluid sample for collection by the collection cartridge. An alternative collection method is also described in the Lauks patent wherein a syringe device is used collect a sample then transfer the sample to the orifice of the cartridge or a reservoir chamber within the cartridge. This method requires additional steps to transfer the collected sample from a syringe to the cartridge which, depending on cartridge size and construction, may be difficult, time consuming and prone to contamination. In addition, as pointed out in the Lauks patent, transfer of exposed blood samples includes the risk of spills, contamination and transmission of infectious diseases such as human immunodeficiency virus or hepatitis.

Therefore, a need exists to provide a cartridge assembly for directly collecting, containing and testing fluid samples such as blood, in association with hand-held analytical devices or stand-alone computer workstations, without requiring exposed sample sources or difficult sample transfers from a collection device to a cartridge reservoir.

### SUMMARY OF THE INVENTION

An object of the present invention is to provide a cartridge assembly which includes an integrated collection mechanism for collecting a fluid sample, and a sample containment chamber for containing and testing collected fluid samples without sample transfers or sample exposure.

This and other objects are substantially achieved by providing a system for collecting a fluid sample, such as blood, directly into a sealed cartridge containing testing mechanisms for evaluation of the collected sample immediately upon collection. The cartridge includes an integrated sample collection mechanism having a shielded piercing element such as a syringe or lancet assembly which is capable of collecting a fluid sample into a containment chamber. The chamber includes an array of electrical contacts, electrochemical sensors and circuitry configured to electrically couple with a hand-held analytical device such as a personal digital assistant (PDA), which controls the testing of the fluid sample within the cartridge and provides a rapid indication of test results.

### BRIEF DESCRIPTION OF THE DRAWINGS

These and other objects, advantages and novel features of the invention will be more readily appreciated from the following detailed description when read in conjunction with the accompanying drawings, in which:

FIG. 1 is a view of an example of one cartridge system including a first integrated syringe assembly collecting mechanism according to an embodiment of the present invention;

FIG. 2 is a view in partial section of the cartridge system of FIG. 1 according to an embodiment of the present invention;

FIG. 3 is a view of an example of one cartridge system including a second integrated syringe assembly collecting mechanism according to an embodiment of the present invention;

FIG. 4 is a view of the cartridge of FIG. 3 engaged with a hand-held analytical device according to an embodiment of the present invention;

FIG. 5 is a view of an example of one cartridge system including an integrated lancet assembly collecting mechanism according to an embodiment of the present invention; and

FIG. 6 is a view of an example of one cartridge system including an integrated lever actuator vacuum assembly collecting mechanism according to an embodiment of the present invention.

In the drawing figures, it will be understood that like numerals refer to like structures and method steps.

### DETAILED DESCRIPTION OF THE PREFERRED EMBODIMENTS

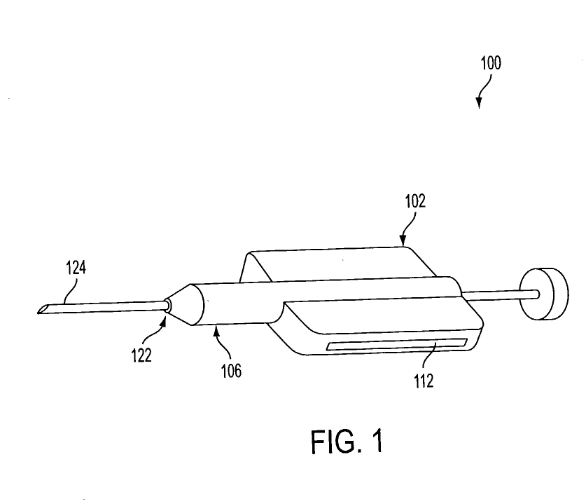

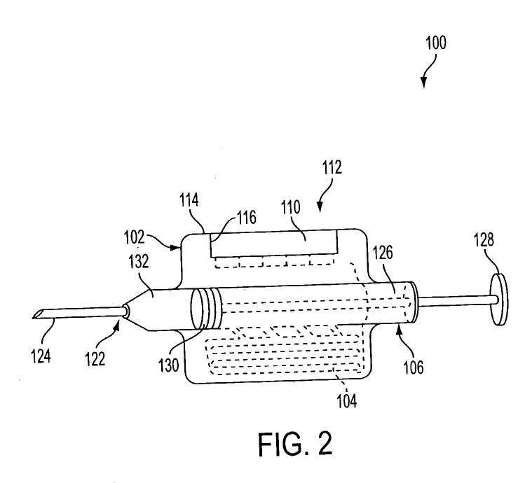

A diagram of an exemplary cartridge collection system 100 in accordance with an embodiment of the present invention is shown in FIGS. 1 and 2. For the following discussion, reference will be made to FIGS. 1 and 2, and as necessary, attention will be drawn to a particular figure. FIGS. 1 and 2 are a view of one example of a cartridge collection system 100, each according to an embodiment of the present invention.

The system 100 of FIG. 1 preferably includes a disposable housing 102 for providing a containment chamber 104 into which fluid samples are placed by an integrated collection mechanism 106 for analysis. The housing 102 is fabricated substantially as a cartridge, with protruding collection mechanism features at opposite ends. For purposes of the description, the housing, including containment chamber and collection mechanism, may be described as a cartridge 102.

As shown in FIG. 2, the collection mechanism 106 located within the cartridge 102 may be used to safely collect a fluid sample into the containment chamber. Once a sample is collected, the collection mechanism may then be shielded and the cartridge may be engaged with a remote analytical device for testing. It will be noted that the embodiments described below include collection mechanisms comprised of needle, lancet, and vacuum assemblies, however, the apparatus of the present invention works just as well with other collection mechanism assemblies.

In FIG. 2, the containment chamber 104 of the cartridge 102 provides contact between collected fluid samples within the chamber and sensory apparatus, such as miniaturized electrodes and micro-sensors, for executing a series of calibration and diagnostic tests on the sample. The sensory apparatus (not shown) is distributed in and around the chamber 104 as known to those skilled in the art, and is electrically coupled to an exposed electrical contact 110 located within a port 112 along the containment chamber wall 114 of the cartridge 102. The exposed electrical contact 110 is contained within a mechanical coupler 116 as known to those skilled in the art, which is adapted to allow direct physical and electrical coupling of the cartridge 102 and a remote analytical device such as a hand-held analyzer, personal digital assistant (PDA) or VISOR@. Alternatively, the cartridge 102 may be coupled with a stand-alone computer workstation, or with any number of existing analyzers, such as those manufactured by the I-Stat Corporation of Princeton N.J.

The electrical contact 110 of cartridge 102 allows the cartridge to engage an input port on a hand-held analytical device and electrically couple the sensory apparatus of the cartridge to a testing and analysis means within the analytical device. Once coupled to the cartridge 102, the analytical device gathers and processes information regarding the contained sample via the sensory apparatus within the containment chamber 104, and thereafter produces outputs which are displayed on an output mechanism, such as a liquid crystal display (LCD), analog display or light emitting diode (LED) indicator. Additional tests on the sample may be directed by activation of user interface mechanisms located on the analytical device. As the cartridge 102 is inexpensive and entirely disposable, upon completion of sample testing the cartridge is removed from the analytical device and discarded.

With reference to the drawings of FIGS. 1 and 2, the system 100 of the embodiment shown comprises a housing 102 having a top and bottom containment wall mechanically coupled to one another in a fashion creating a chamber 104 providing for the capture and containment of a sample substance such as blood, from an integrated collection mechanism 106. The housing, or cartridge, 102 has a distal end with a collection mechanism inlet orifice 122 formed therein, the orifice 122 being adapted to engage an integrated piercing element 124, forming a leak-proof seal between orifice and element. The cartridge 102 also has a proximal end having a plunger assembly orifice formed therein, allowing external control of the plunger assembly 126.

As used herein, the term “proximal” refers to a location on the housing 102 closest to the person using the device and farthest from the patient in connection with which the device is used. Conversely, the term “distal” refers to a location on the housing 102 farthest from the person using the device and closest to the patient in connection with which the device is used.

As known by those skilled in the art, a fluid sample may be easily collected by a collection mechanism 106, where the integrated piercing element 124 is an intra-venous needle, intra-arterial needle, venous catheter, arterial catheter, capillary tube, microneedle array or lancet. One embodiment of an integrated collection mechanism 106 shown in FIGS. 1 and 2 includes a plunger assembly 126, which extends through the proximal end of the cartridge 102 and into a body cavity 132, wherein the plunger assembly controls a piston 130 to slide in a fluid-tight engagement within the walls of the cavity 132. The plunger assembly 126 is mechanically coupled to an external mechanism 128 for moving the plunger within the cavity 132, creating a vacuum within the cavity for drawing a fluid sample into the cavity via the inlet orifice 122. The inlet orifice 122 can also include a back-flow prevention mechanism, such as a ball or gate valve (not shown), to prevent the fluid sample from escaping during testing.

In fluid communication with the body cavity 132, a containment chamber 104 is positioned about the body cavity such that sufficient fluid is communicated across the sensory apparatus located in and around the chamber 104. As shown in FIG. 2, fluid communication between body cavity 132 and chamber 104 can be achieved via multiple ports between the two, however other embodiments can use fewer ports, or conjoined ports, resulting in a single slot between body cavity and chamber. The port(s) create a path between the body cavity 132 and chamber 104 which allows the capillary action of the ports and chamber to communicate a fluid sample from the body cavity. The capillary action of the containment chamber 104 completes the capture of the fluid sample, which is then communicated throughout the chamber.

The ports can be positioned to maximize fluid communication, and utilize any number of methods, such as capillary action or the vacuum created by the slidable engagement of the plunger assembly 126, to communicate a fluid sample across the sensory apparatus of the chamber 104. The port configuration allows cavity 132 and chamber 104 to function as a single repository for sample collection.

Once a sufficient sample is captured and contained within the cartridge 102 and communicated throughout chamber 104, the integrated piercing element 124 maybe shielded or retracted as known to those skilled in the art by activating a shielding mechanism (not shown). Existing safety-engineered sharps protection systems include shields that pivot over needles, needles that retract into shields, and shields that move forward, relative to the needle, in order to contain the point of the needle. Needle pivoting, shielding or retracting systems and methods are widely practiced and are described in numerous documents, such as U.S. Pat. No. 6,368,303 issued to Richard Caizza, and in U.S. Pat. No. 6,319,232 issued to James Kashmer, the entire contents of each being incorporated herein by reference. Furthermore, the integrated collection mechanism 106 may include a number of different lancet and needle assemblies as required by the specific application.

The system 100 of FIG. 1 further includes sensory apparatus within the cartridge 102 and in communication with the collected sample within the containment chamber 104. As can be appreciated by one skilled in the art, the sensory apparatus may consist of a number of miniaturized electrodes and biosensors adapted to detect and measure various chemical and physical properties of the sample contained within the chamber. The sensory apparatus is distributed in and around the chamber 104 of the cartridge 102 and is electrically coupled to the exposed electrical contact 110 located externally along the containment chamber wall 114 of the cartridge 102. The exposed electrical contact 110 is contained within a mechanical coupler 116 adapted to allow direct physical and electrical coupling of the cartridge 102 and a like electrical contact and mechanical coupler located on a remote analytical device, such as a hand-held analyzer, personal digital assistant (PDA) or VISOR®, or stand-alone computer workstation. Once within the containment chamber 104, the sample is subject to extensive analysis through direct and indirect contacts with the sensory apparatus, as directed by the remote analytical device.

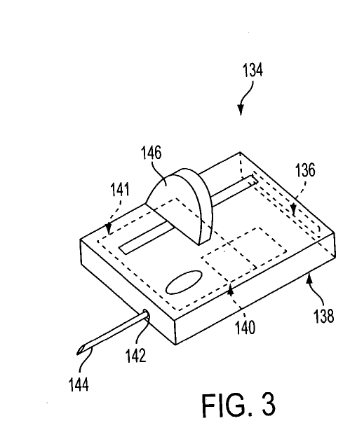

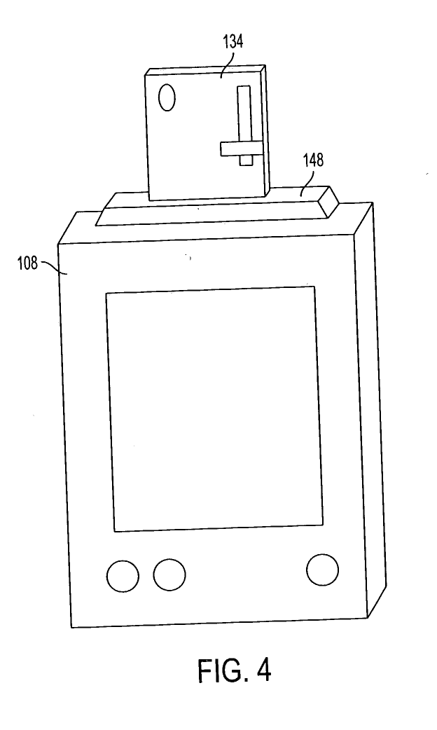

In another embodiment of the present invention, a diagram of an exemplary system 134 is shown in FIGS. 3 and 4. For the following discussion, reference will be made to FIGS. 3 and 4, and as necessary, attention will be drawn to a particular figure. FIG. 3 is a view in partial section of one example of a cartridge collection system 134, and FIG. 4 is a view of the cartridge of FIG. 3 engaged with a hand-held analytical device, each according to an embodiment of the present invention.

The system 134 of FIG. 3 preferably includes a disposable cartridge 136 for providing a containment chamber 140 into which fluid samples are placed by an integrated collection mechanism 141 for analysis substantially as described above. Once a sample is collected into the cartridge, the collection mechanism 141 may then be shielded and the cartridge may be engaged with a remote analytical device 108 for testing as shown in FIG. 4.

With reference to the drawings of FIGS. 3 and 4, the system 134 of the embodiment shown comprises a housing, or cartridge 138, having a top and bottom containment wall mechanically coupled to one another in a fashion creating a chamber 140 providing for the capture and containment of a sample substance such as blood, from an integrated collection mechanism 141. The cartridge 138 has a distal end with a collection mechanism inlet orifice 142 formed therein, the orifice 142 being adapted to engage an integrated piercing element 144. While engaged, a leak-proof seal is formed between the inlet orifice 142 and the integrated piercing element 144. The cartridge 138 also has a proximal end having an exposed electrical contact 136 formed therein.

As known by those skilled in the art, a fluid sample may be easily collected by a collection mechanism 141, where the mechanism is comprised of a syringe assembly contained within the cartridge 138. The collection mechanism 141 is controlled by an external operator interface 146, such as a lever, mechanically coupled to a plunger assembly for moving the plunger within the containment chamber 140, creating a vacuum within the chamber for drawing a fluid sample into the chamber via the inlet orifice 142. Once a sufficient sample is captured and contained within the cartridge 138 and communicated throughout chamber, the integrated piercing element 144 maybe shielded or retracted. The exposed electrical contact 136 is located opposite the distal end of the cartridge 138 as a convenience and safety feature. During coupling of the cartridge with the analytical device, operator activity is focused about the proximal end, away from the sharp distal end.

Sample testing with the preferred embodiments of the present invention is achieved by first collecting a fluid sample from a patient using the integrated collection mechanism as known to those skilled in the art and thereafter, the piercing element of the integrated collection mechanism may be retracted or shielded. The fluid sample is positioned within the containment chamber and is in communication with the sensor array distributed about the cartridge. A remote analytical device 108 is then prepared to receive the filled cartridge 102 or 138 for testing of the contained fluid sample.

As known to those skilled in the art, many interface modules are provided to adapt hand-held devices for multiple uses, such as SPRINGBOARD® expansion modules for a personal digital assistant (PDA) or VISOR® as shown in FIG. 4. Where required, an interface module 148 is installed on the analytical device 108 which allows engagement with the exposed electrical contact and mechanical coupler of the cartridge 102 and 138. As appreciated by those skilled in the art, the analytical device 108 includes hardware and software adapted to access the sensory apparatus within the cartridge and gather information on the sample contained therein, such as pH, pCO₂, pO₂ Na⁺, Ca⁺⁺, K⁺, hematocrit and glucose levels in the sample, in addition to sample temperature measurements. The analytical device may then perform numerous tests, configured by the user, on the sample contained within the containment chamber of the cartridge and display results via an output mechanism, such as a liquid crystal display (LCD), analog display or light emitting diode (LED) indicator on the analytical device. Additional tests or property detection may be directed by activation of user interface mechanisms located on the analytical device. Upon completion, the cartridge and sample therein, are removed from the analytical device and disposed. The cartridge is fabricated as a sterile, disposable unit, preferably made of an inexpensive plastic, such as polyethylene, polypropylene, polyvinylidene chloride or the like. Additionally, the sensory apparatus within the cartridge are sufficiently inexpensive to allow single use and disposal.

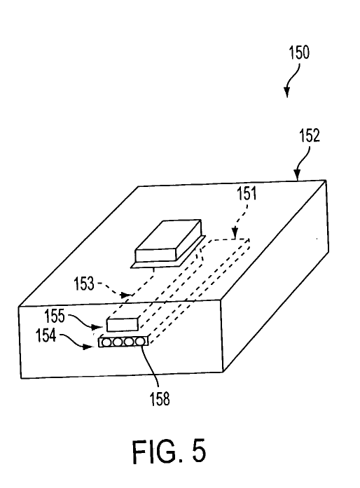

In another embodiment of the present invention, a diagram of an exemplary system 150 is shown in FIG. 5. FIG. 5 is a view in partial section of one example of a cartridge collection system 150 and preferably includes a disposable cartridge 152 and a containment chamber 151 into which fluid samples are placed for analysis, substantially the same as discussed above for the collection cartridge 102. The cartridge system 150 includes an integrated lancet assembly collection mechanism 153, which may be activated and used to draw a fluid sample to the surface of a patient's skin for collection directly into the containment chamber of the cartridge.

The system 150 of FIG. 5 comprises a cartridge 152 having a containment chamber 151 and a lancet mechanism 153 positioned to operate at a point about the distal end of the cartridge. The cartridge 152 includes a containment chamber inlet orifice array 154 described below, adjacent to the lancet mechanism piercing assembly 155, to allow collection of exposed fluid samples.

With reference to the drawing of FIG. 5, the cartridge 152 includes a top and bottom containment wall, mechanically coupled to one another in a fashion creating a containment chamber 151 providing for the capture and containment of a sample substance. The cartridge 152 has a distal end with a containment chamber inlet orifice array 154 formed therein. The orifice array 154 includes a number of inlet passageways 158 extending through the housing 152 allowing access to the containment chamber 151 within the cartridge 150. The containment chamber of the cartridge provides contact between collected fluid samples within the chamber and sensory apparatus for executing a series of calibration and diagnostic tests on the sample substantially the same as discussed above for the collection cartridge 102.

As known by those skilled in the art, a lancet may be used to draw a blood sample from a patient. In the embodiment shown in FIG. 5, the integrated lancet assembly collection mechanism 153 and lancet mechanism piercing assembly 155 are used to expose a fluid sample from a patient. The orifice array 154, adjacent to the assembly 155 contacts the exposed fluid and communicates a sample into the containment chamber 151 via the capillary action of a number of inlet passageways 158 extending through the housing 152 and the containment chamber 151 within the cartridge 150. Once a sufficient sample is captured and contained within the cartridge 152 and communicated throughout chamber 151, the lancet mechanism piercing assembly 155 maybe shielded or retracted as known to those skilled in the art by activating a shielding mechanism (not shown). The sample is then tested as described above. Additional details of lancet use are described in U.S. Pat. No. 4,677,979 issued to James A. Burns, the entire content of which is incorporated herein by reference. The cartridge 150 allows the collection of a portion of the drawn fluid sample into the containment chamber of the cartridge via the inlet orifice array 154 using methods such as the capillary action of the inlet passageways 158. Other methods, such as a vacuum may also be used to draw the sample into the containment chamber.

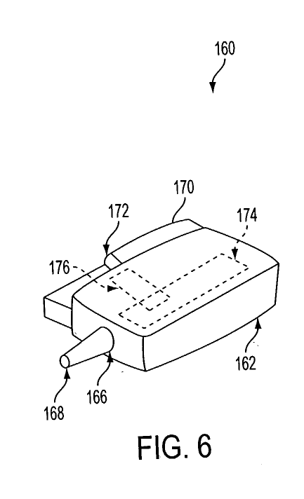

In yet another embodiment, the collection mechanism may include a lever actuator vacuum assembly to draw a sample into the containment chamber 174. In the embodiment shown in FIG. 6, the collection cartridge system 160 of the embodiment shown comprises a housing 162 having a top and bottom containment wall, mechanically coupled to one another in a fashion creating a containment chamber 174 providing for the capture and containment of a sample substance substantially the same as discussed above for the collection cartridge 102. The housing 162 has a distal end with a containment chamber inlet orifice 166 formed therein. The orifice 166 includes a conical luer fitting 168 extending from the housing 162 allowing access to the containment chamber 174 within the cartridge. The luer fitting 168 may also be used to attach an intra venous or intra-arterial needle to the cartridge.

The cartridge 160 allows the collection of a fluid sample into the containment chamber 174 via the inlet orifice 166 using a vacuum created by an integrated vacuum assembly within the housing 162. An external lever 170 may be rotationally moved from a first position to a second position about an axis 172 driving an internal assembly 176 creating a vacuum within the containment chamber 174 as known to those skilled in the art. The containment chamber of the cartridge provides contact between collected fluid samples within the chamber and sensory apparatus for executing a series of calibration and diagnostic tests on the sample substantially the same as discussed above for the collection cartridge 102.

The collection cartridge disclosed in each embodiment provides the ability to obtain immediate, reliable and accurate testing of fluid samples without the processing delays associated with traditional laboratories. Moreover, the cartridge greatly reduces the quantity of fluid sample required from the patient to perform these tests.

Although only a few exemplary embodiments of the present invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims.

What is claimed is:

1. An integrated collection device for collecting fluid samples and performing fluid sample analysis, comprising:
a housing comprising an inlet orifice and a top and bottom containment wall, said top wall mechanically coupled to said bottom wall to define a containment chamber, wherein said containment chamber is accessible through said inlet orifice, said housing further having an integrated collection mechanism and an electrical connector;
said integrated collection mechanism comprising a distal port and a body cavity in fluid communication with said distal port adapted to collect fluid from a patient and communicate a fluid sample of said collected fluid into said containment chamber via said inlet orifice;
said electrical connector of said housing comprising a first set of electrical contacts adapted to engage a second set of electrical contacts located on an analytical device, said first set of electrical contacts electrically coupled to a sensory apparatus disposed about said containment chamber; and
said sensory apparatus comprising at least one sensor adapted to detect fluid properties, said sensory apparatus electrically coupled to said first set of electrical contacts and accessible by said analytical device via said electrical connector.

2. An integrated collection device for collecting fluid samples and performing fluid sample analysis as claimed in claim 1, wherein said distal port of said integrated collection mechanism comprises a collection element engagement mechanism.

3. An integrated collection device for collecting fluid samples and performing fluid sample analysis as claimed in claim 2, wherein said collection element engagement mechanism comprises a conical luer fitting.

4. An integrated collection device for collecting fluid samples and performing fluid sample analysis as claimed in claim 2, wherein said collection element is selected from the group consisting of an intra-venous needle, intra-arterial needle, venous catheter, arterial catheter, capillary tube, microneedle array and lancet.

5. An integrated collection device for collecting fluid samples and performing fluid sample analysis as claimed in claim 2, wherein said collection element engagement mechanism comprises a shielding mechanism adapted to shield said collection element.

6. An integrated collection device for collecting fluid samples and performing fluid sample analysis as claimed in claim 5, wherein said housing further comprises a first user interface control to control said shielding mechanism.

7. An integrated collection device for collecting fluid samples and performing fluid sample analysis as claimed in claim 1, wherein said integrated collection mechanism is adapted to create a vacuum and in response, said distal port is adapted to collect fluid from a patient into said body cavity and communicate said fluid sample of said collected fluid into said containment chamber.

8. An integrated collection device for collecting fluid samples and performing fluid sample analysis as claimed in claim 7, wherein said integrated collection mechanism further comprises a plunger assembly slidably engaged within said body cavity wherein said movement of said plunger assembly within said body cavity creates said vacuum.

9. An integrated collection device for collecting fluid samples and performing fluid sample analysis as claimed in claim 7, wherein said distal port is adapted to communicate said fluid sample of said collected fluid into said containment chamber via a capillary action created by at least one of said inlet orifice and said containment chamber.

10. An integrated collection device for collecting fluid samples and performing fluid sample analysis as claimed in claim 7, wherein said distal port is adapted to communicate said fluid sample of said collected fluid into said containment chamber via said vacuum created by said integrated collection mechanism.

11. An integrated collection device for collecting fluid samples and performing fluid sample analysis as claimed in claim 8, wherein said plunger assembly comprises a second user interface control to control said movement of said plunger assembly.

12. An integrated collection device for collecting fluid samples and performing fluid sample analysis as claimed in claim 1, wherein said integrated collection mechanism is adapted to expose fluid from a patient and in response, said distal port is adapted to collect fluid from a patient into said body cavity and communicate said fluid sample of said collected fluid into said containment chamber.

13. An integrated collection device for collecting fluid samples and performing fluid sample analysis as claimed in claim 12, wherein said distal port is adapted to communicate said fluid sample of said collected fluid into said containment chamber via a capillary action created by at least one of said distal port, body cavity, inlet orifice and said containment chamber.

14. An integrated collection device for collecting fluid samples and performing fluid sample analysis as claimed in claim 1, wherein said second electrical connector comprises a second set of electrical contacts, said second electrical connector adapted to engage said first electrical connector of said collection cartridge and electrically couple said first and second set of electrical contacts.

15. An integrated collection device for collecting fluid samples and performing fluid sample analysis as claimed in claim 1, wherein said analytical device is adapted to perform user directed analytical tests on said fluid sample using said sensory apparatus via said first and second sets of electrical contacts.

16. An integrated collection device for collecting fluid samples and performing fluid sample analysis as claimed in claim 1, wherein said analytical device is selected from the group consisting of a hand-held analyzer, personal digital assistant and computer workstation.

17. A cartridge assembly for collecting fluid samples and performing fluid sample analysis as claimed in claim 1, wherein said fluid sample properties include at least one of a pH, pCO₂, pO₂, pCl, pNO₃, Na⁺, Ca⁺⁺, K⁺, hematocrit and glucose level in said sample.

18. A cartridge assembly for collecting fluid samples and performing fluid sample analysis as claimed in claim 1, wherein said fluid sample properties includes a temperature of said fluid sample.
